(19) United States
(12) Patent Application Publication
(10) Pub. No.: US 2023/0211100 A1
(43) Pub. Date: Jul. 6, 2023

(54) SYSTEM AND METHOD FOR PREDICTIVE WEANING OF VENTILATED PATIENTS

(71) Applicant: Vyaire Medical, Inc., Mettawa, IL (US)

(72) Inventors: Alejandro Jose VILLASMIL, Irvine, CA (US); Christopher M. VARGA, Palm Coast, FL (US); Hasnain SOMJI, Irvine, CA (US)

(21) Appl. No.: 17/928,261

(22) PCT Filed: May 27, 2021
(86) PCT No.: PCT/US2021/034595

§ 371 Date: Nov. 28, 2022

Related U.S. Application Data
(60) Provisional application No. 63/031,489, filed on May 28, 2020.

(51) Int. Cl. A61M 16/00; G16H 50/70; G16H 40/40

(52) U.S. Cl. CPC A61M 16/026; G16H 50/70; G16H 40/40; A61M 2230/40; A61M 2205/3303

(57) ABSTRACT

The disclosed system and method generates a trained prediction model based on a plurality of sets of sampled ventilation parameter values received from patient ventilations, and a plurality of weaning indicators representative of patient outcomes for each sampled patient ventilation. Ventilation parameter values are sampled during a current patient ventilation and input into the trained prediction model. The model selects, from the group of ventilation parameters, a ventilation parameter and associated parameter value or range of parameter values having the highest probability of positively influencing the current patient ventilation based on a threshold value of the ventilator parameter. The system may then use the returned parameter value(s) to cause an operational mode of a ventilator associated with the current patient ventilation to be adjusted.

### CROSS REFERENCE TO RELATED APPLICATIONS

This application claims the benefit of U.S. Provisional Application Ser. No. 63/031,489, entitled “SYSTEM AND METHOD FOR PREDICTIVE WEANING OF VENTILATED PATIENTS,” filed on May 28, 2020, the entirety of which is incorporated herein by reference.

### BACKGROUND FIELD

The subject technology addresses deficiencies commonly encountered in hospital care with regard to assessing conditions of ventilated patients and adjusting ventilation parameters to stabilize and wean such patients from ventilation.

### SUMMARY

A mechanical ventilator provides life support by assisting the patient with the inhalation of oxygen and the exhalation of CO2 in order to maintain the necessary PaO2, PaCo2 and pH arterial blood levels, when the patient is unable to sustain adequate levels with their own spontaneous breathing. Positive pressure mechanical ventilators pump air with a controllable percentage of inspired oxygen during the inspiratory phase of the breathing cycle. When the inspiratory phase of the breathing cycle is complete, the patient exhales through the ventilator by utilizing the natural recoil characteristics of the lungs. The volume of air that is introduced into the lungs on each cycle is the “tidal volume.” This process is very invasive and introduces a high potential for complications such as barotrauma and secondary infections. Furthermore, the analgesics (or other pain medication) and sedatives commonly prescribed to such patients to provide patient comfort can themselves lead to adverse patient outcomes

Thus, it is desirable to end the use of a mechanical ventilator as early as possible. Many of the rules and protocols for transitioning a patient off of a mechanical ventilator, or “weaning” the patient, include a series of clinical interventions including an adjustment to an amount of sedatives or analgesics to awaken the patient, in addition to reducing or stopping ventilation support for a period of time, all while monitoring the patient to identify signs of distress or difficulty. If the patient is able to successfully complete the prescribed weaning trials, “extubation” may be performed where the invasive ventilator support is removed, or the patient may be put back on full support to further prepare them for extubation.

Physician extubation decision accuracy has been shown to be low. And, increased duration of Intensive Care Unit (ICU) mechanical ventilation is associated with negative outcomes ranging from increased incidence of Ventilator Associated Events (VAE) and Acute Respiratory Distress Syndrome (ARDS), to higher mortality, healthcare utilization, and costs. Prediction of extubation candidacy as early and as accurately as possible in the course of ventilated patient care is therefore desirable.

Additionally, current physician extubation candidacy predictions are subjective and vary significantly from professional to professional. The disclosed system provides objectivity for extubation procedures and enables a standardized approach to reduce or eliminate variability which is otherwise present from physician to physician.

The subject technology addresses deficiencies commonly encountered in hospital care and medical care involving assessment of mechanically ventilated patient status with respect to ventilator weaning and patient extubation candidacy, and thus increases the accuracy and reliability of the critical decision making process. The disclosed system of the subject technology may provide twice the accuracy of physician predictions in identifying patients who are ready to be extubated, and three times the accuracy of physician predictions in identifying patients who are not ready to be extubated. The system thus bridges accuracy gaps present in the Medical ICU.

According to various implementations, the disclosed system includes one or more processors and a memory. The memory includes instructions stored thereon that, when executed by the one or more processors, cause the one or more processors to perform operations for performing a method for predictive weaning or extubation of a ventilated patient, and adjusting an operation mode of the ventilator to accomplish this result. The method includes receiving, by one or more computing devices, a plurality of sets of sampled ventilation parameter values for a plurality of patient ventilations in a patient population, each patient ventilation being associated with a group of ventilation parameters for which one or more of the plurality of sets is sampled during a same time period; receiving, by the one or more computing devices, a plurality of weaning indicators, each of the plurality of weaning indicators corresponding to a respective patient ventilation of the plurality of patient ventilations and the one or more of the plurality of sets sampled during the same time period associated with the respective patient ventilation; generating, by the one or more computing devices, a trained prediction model based on the received plurality of sets of sampled ventilation parameter values and the received plurality of weaning indicators, the trained prediction model being trained to select, based on an input of ventilation parameter values for a patient ventilation, one or more ventilator parameters having a highest probability, within the group of ventilation parameters, of positively influencing the patient ventilation based on respective threshold values; receiving, by the one or more computing devices, a plurality of ventilation parameter values sampled during a current patient ventilation; automatically inputting, by the one or more computing devices, the plurality of ventilation parameter values sampled during the current patient ventilation into the trained prediction model; receiving, from the trained prediction model, by the one or more computing devices, based on the inputting of the plurality of ventilation parameters, a ventilation parameter selected from the group of ventilation parameters and having the highest probability of positively influencing the current patient ventilation based on a threshold value of the ventilator parameter; and causing, by the one or more computing devices, an operational mode of a ventilator associated with the current patient ventilation to be adjusted based on the ventilation parameter selected by the trained prediction model. Other aspects include corresponding systems, apparatuses, and computer program products for implementation of the computer-implemented method.

Further aspects of the subject technology, features, and advantages, as well as the structure and operation of various aspects of the subject technology are described in detail below with reference to accompanying drawings.

### DESCRIPTION OF THE FIGURES

Various objects, features, and advantages of the present disclosure can be more fully appreciated with reference to the following detailed description when considered in connection with the following drawings, in which like reference numerals identify like elements. The following drawings are for the purpose of illustration only and are not intended to be limiting of this disclosure, the scope of which is set forth in the claims that follow.

FIG. 1 depicts an example ventilation feature matrix structure for training predictive models, according to various aspects of the subject technology.

FIG. 2 depicts an example workflow diagram for training and testing a predictive model, according to various aspects of the subject technology.

FIG. 3 depicts first example performance metrics resulting from trained predictive models, according to various aspects of the subject technology.

FIG. 4 depicts second example performance metrics resulting from trained predictive models, according to various aspects of the subject technology.

FIG. 5 depicts an example feature weighting and comparison for determining feature importance in a predictive model, according to various aspects of the subject technology.

FIGS. 6A, 6B, and 6C depict example dependence plots for an effect of different ventilation parameters on extubation probability, as predicted by the disclosed system, according to various aspects of the subject technology.

FIG. 7 depicts an example two-dimensional partial dependence plot illustrating a correlation between two ventilation parameters with regard to their effect on extubation probability, according to various aspects of the subject technology.

FIG. 8 depicts an example closed-loop system incorporating an automated weaning and extubation model, according to various aspects of the subject technology.

FIG. 9 depicts a flow diagram of an example extubation protocol tailored based on patient reintubation risk, according to various aspects of the subject technology.

FIG. 10 depicts an example closed-loop system incorporating an automated weaning and extubation model, tailored based on patient reintubation risk, according to various aspects of the subject technology.

FIG. 11 is a block diagram illustrating an example system for predictive weaning of ventilated patients, including multiple ventilation devices and a ventilation management system, according to certain aspects of the subject technology.

FIG. 12 depicts an example flow chart of a method of generating patient-specific ventilation settings based on a trained prediction model, and for adjusting an operation of a ventilator to wean a patient from ventilation, according to aspects of the subject technology.

FIG. 13 is a conceptual diagram illustrating an example electronic system for generating patient-specific ventilation settings based on a trained prediction model, and for adjusting an operation of a ventilator, according to aspects of the subject technology.

### DESCRIPTION

While aspects of the subject technology are described herein with reference to illustrative examples for particular applications, it should be understood that the subject technology is not limited to those particular applications. Those skilled in the art with access to the teachings provided herein will recognize additional modifications, applications, and aspects within the scope thereof and additional fields in which the subject technology would be of significant utility.

The subject technology comprises a computer-enabled system which generates statistical and machine learning models from ventilator-recorded parameters and inputs obtained from integrated measurement devices and components. In some implementations, the system of the subject technology utilizes the models and parameter inputs to predict extubation candidacy and provide patient-specific optimal ventilation settings, modes, or options.

The ventilator-recorded parameters may include one or more of: measurement(s) indicating compliance of the lung (Cdyn, Cstat), dynamic compliance, flow resistance of the patient airways (Raw), inspiratory-expiratory ratio (FE), spontaneous ventilation rate, exhaled tidal volume (Vte), total lung ventilation per minute (Ve), peak expiratory flow rate (PEFR), peak inspiratory flow rate (PIFR), expiratory time, inspiratory time, mean airway pressure, peak airway pressure, breathing rate, mandatory or spontaneous breathing rate, an average end-tidal expired CO2 and total ventilation rate, a ventilation mode, a set mandatory or inspiratory tidal volume, spontaneous minute volume, total minute volume, exhaled tidal volume, spontaneous exhaled tidal volume, vent work of breathing, positive-end expiratory pressure (PEEP), an apnea interval, a bias flow, a breathing circuit compressible volume, a patient airway type (for example endotracheal tube, tracheostomy tube, face mask) and size, a fraction of inspired oxygen (FiO2), a breath cycle threshold, and a breath trigger threshold. Other measured inputs may include, for example, objective patient physiological attributes, such as oxygen levels, blood pressure, and the like. As will be described further, one or more of these time-series parameters are utilized to train machine learning models to predict extubation candidacy a period of time (e.g., two hours) in advance of extubation.

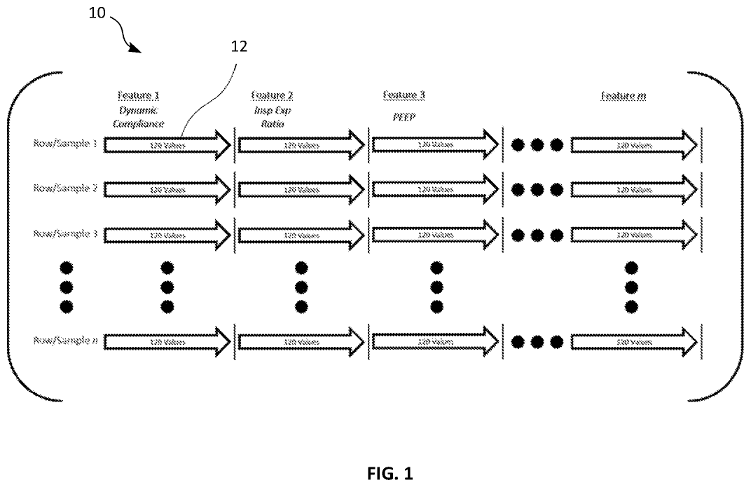

FIG. 1 depicts an example ventilation feature matrix structure 10 for training predictive models, according to various aspects of the subject technology. The system of the subject technology utilizes a pre-processing pipeline utilized and a predictive learning algorithm to train one or more predictive models based on the foregoing ventilator-recorded data. The pre-processing pipeline is independent of the learning algorithm. Time-series ventilator parameters such as those listed above are imported as a matrix, with each row corresponding to samples for a particular patient, and each column 12 within each row corresponding to one of a set of sampled parameters. A linear interpolation may be implemented in order to mitigate the presence of any sparse missing values in the dataset.

Each sampled parameter 12 within a row is represented by a group or series of samples over a predetermined period. This group or series of samples is what makes up a particular column within matrix 10. Sampling periods may range in time from seconds to minutes to hours. For example, the sampling period may be 1 minute. Accordingly, each group of samples (e.g., a column) may include a series of 1 minute samples over a given period, for example, over a two hour period of time. Each row may also include a corresponding time-stamp reflecting the time in which the samples within that row were recorded.

Thereafter, a one-dimensional vector containing binary labels (1 or 0), each corresponding to whether a given row of data resulted in a positive or negative extubation outcome, may be combined with the foregoing data. In this regard, data may be collected regarding whether a given patient resolved or became an extubation candidate during the same time period in which the ventilation parameters were sampled. Each value (or “extubation label”) of the one-dimensional vector may correspond to this time period. When combined with the foregoing sample data, the extubation label indicates whether, during the corresponding time period, the patient became a candidate for extubation.

Feature matrix 10 is generated for use as a training data set from the ventilator monitored parameter data and the corresponding labels. Feature matrix 10 comprises a number of rows corresponding to a given number of patients in a patient population, and a number of columns corresponding to time-series ventilator parameters sampled during treatment of those patients. For example, each row may pertain to an individual patient. Although, in some implementations, multiple rows may be used for a single patient, or multiple patients may be represented by a single row. The number of columns used may be dependent on the number of features to be taken into consideration in the predictive algorithm, and the preferred time window length for sampling selected based on a desired accuracy for making real-time predictions.

In the depicted example, matrix 10 includes n rows and m features (e.g., parameters), with each feature including 120 sample values. For example, in some implementations, 19 features are used, with a time window of 2 hours and a sampling rate of 1 sample per minute, providing 120 samples per feature, leading to a feature matrix with 2280 columns. In this example, a one-dimensional extubation label vector containing 1s or 0s is created, and matched with each sample in the feature matrix based on respective timestamps and/or feature sampling periods.

The system (e.g., via the pre-processing pipeline) generates feature matrix 10 with an amount of rows corresponding to a total number of patients, and an amount of columns corresponding to the product of the number of utilized ventilator features and the number of samples (e.g., minutes) within a given time window. As described previously, in some implementations, the number of rows may not be equal to the number of patients but rather, a single row may include multiple patients or a single patient may be represented by data spanning multiple rows. The extubation label vector possesses the same number of rows as the feature matrix such that every sample has a corresponding positive or negative label signaling whether or not a patient is considered an extubation candidate. Given these two components, the predictive models can be trained.

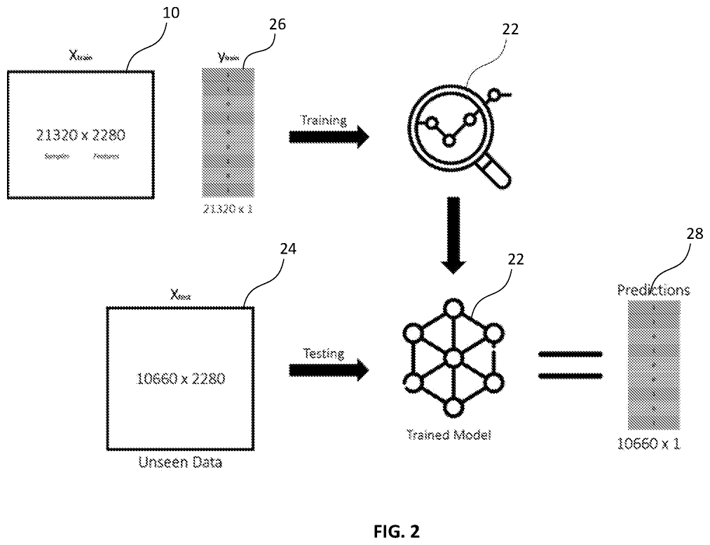

FIG. 2 depicts an example workflow diagram for training and testing a predictive model, according to various aspects of the subject technology. According to various implementations, a predictive model 22 is trained using feature matrix 10 (including extubation label vector). According to various implementations, one or more predictive models may be used. Four exemplary models include Multi-Layer Perceptron Artificial Neural Network, Random Forest Ensemble, Logistic Regression Model, and Support Vector Machine Classifier. Other predictive models may also be used. The models may be trained iteratively using the feature matrix 10 as training data, as described above, to minimize a loss function, which gauges the discrepancies in the training predictions and actual label values present in the extubation label vector. In some implementations, generalization of the algorithms to unseen data is gauged using 10-fold cross-validation applied to a training data set. In these implementations, a training data set may be shuffled and split into 10 folds, and training is subsequently executed on 9 folds to generate predictions on the 10th fold. This process can be repeated multiple times to generate predictions for the entire training data set. For example, this process may be repeated ten times.

Once initially trained, the model may be used by the predictive algorithm to determine success on unseen data 24 (without having to use a further training data set). In other words, the trained model can be used to generate predictions on an unseen testing set of ventilator monitored parameter data. Training may be deemed complete when a predetermined level of success is obtained with regard to the unseen data 24. That is, while a one-dimensional vector of extubation labels 26 may be provided as part of matrix 10, a one-dimensional vector of extubation labels for the unseen data may be set aside and then later compared to prediction results 26 made by the model. The model may be considered to be adequately trained when the success rate, based on this comparison, reaches the threshold.

Feature matrix 10 may include one or more ventilator time-series parameters (e.g. 19), a time window (e.g. 120 minutes), and extubation labels, to provide extubation predictions on test data sets. Performance metrics such as accuracy, precision, F-score, and AUC for the Receiver Operating Characteristic (ROC) curve, may be determined to further evaluate success of the testing set extubation predictions against the true testing set extubation labels.

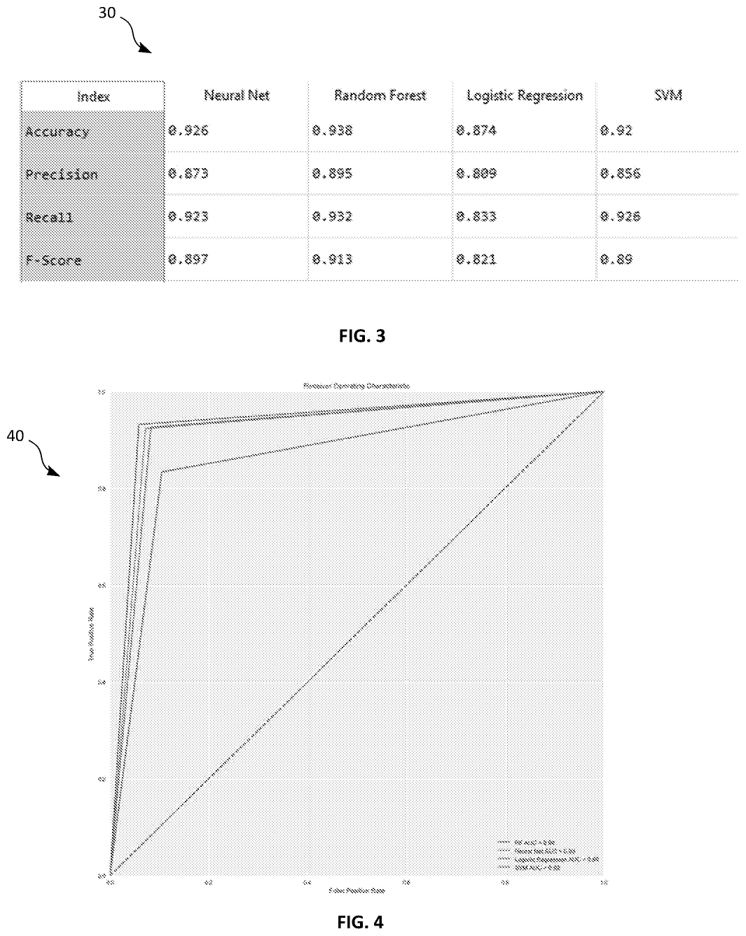

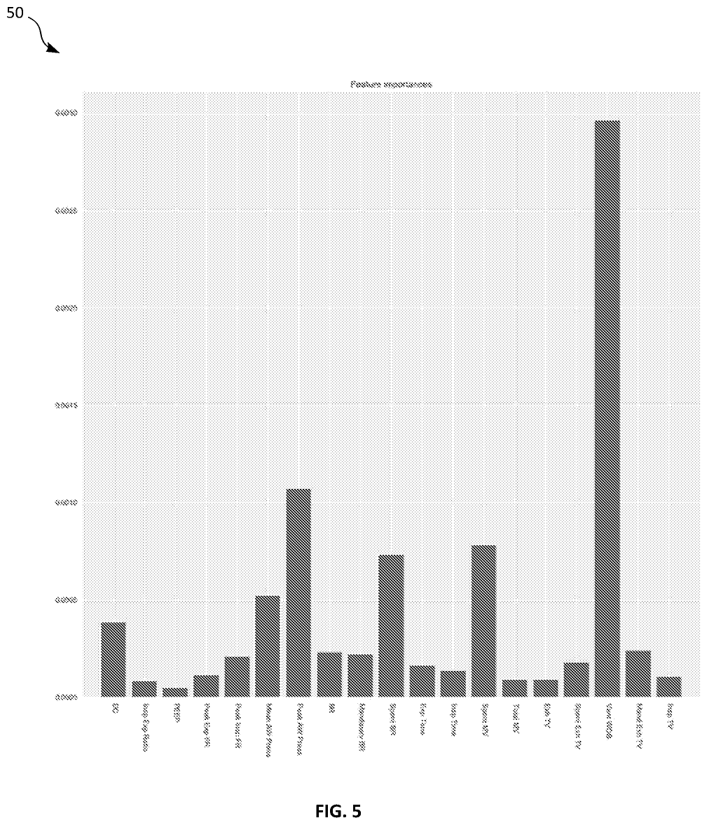

FIG. 3 and FIG. 4 depict example performance metrics resulting from trained predictive models, according to various aspects of the subject technology. It can be observed in the depicted results that each predictive model may produce a varying level of accuracy, precision, recall and F-score. In the depicted example 40, the Random Forest Ensemble model produces a F-score of 0.913, an accuracy of 0.938 and an AUC of 0.94 while the Deep Neural Network model produces a F-score of 0.897, an accuracy of 0.926, and an AUC of 0.93. It should be appreciated that these results are specific to the particular set of ventilation parameters (e.g. 19) leveraged but these results may vary with different numbers or types of ventilation parameters. Amongst the previously described model types, with the given set of ventilation time-series parameters, the Random Forest and Deep Neural Network embodiments/models provided the most accurate and reliable extubation candidacy predictions for the given example data set.

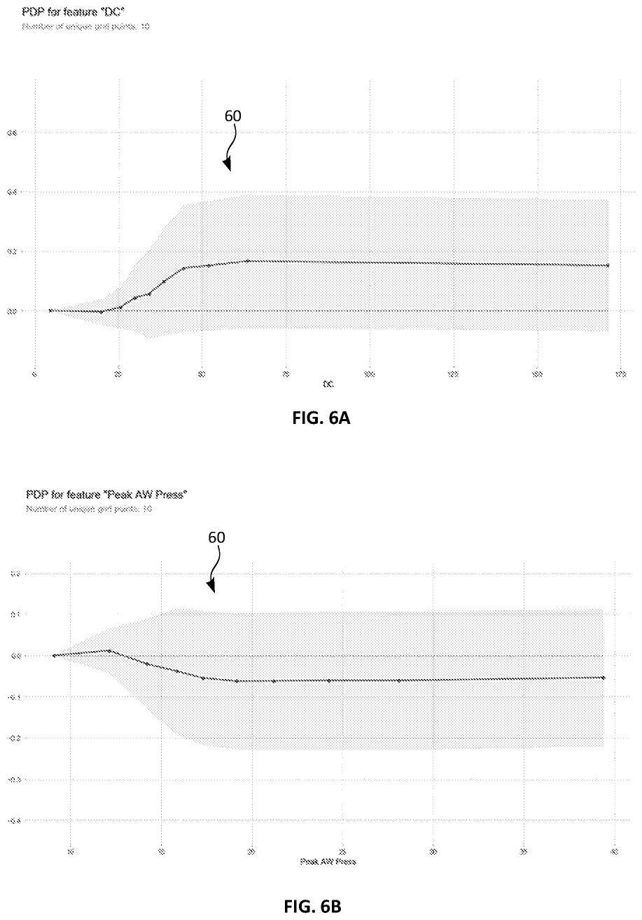

FIG. 5 depicts an example feature weighting and comparison for determining feature importance in a predictive model, according to various aspects of the subject technology. The depicted example is based on the results of the Random Forest predictive model. The parameters of ventilator work-of-breathing (WOB), peak airway pressure, and spontaneous minute volume were determined to be the most informative to predicting extubation success, while dynamic compliance and spontaneous breath rate are also important features. These features carry the bulk of the information used to determine whether a patient sample is deemed as a positive or negative candidate.

Models trained according to the subject technology provide the disclosed system with the sensitivity to repeatedly detect instances in which a given patient is ready for extubation, and also to predict when a patient is not ready for extubation. In one study, the accuracy of physicians extubation decisions had an AUC of the Receiver Operating Characteristic Curve of 0.35 which, for the same available data, can be compared to values of 0.93 and 0.94 produced by the system and model of the subject technology. Accordingly, implementation of the subject technology in a clinical setting may facility the mitigation of negative outcomes associated with increased duration of mechanical ventilation.

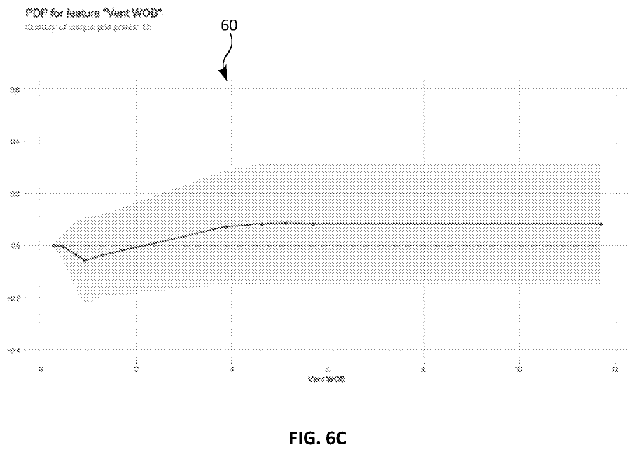

FIGS. 6A, 6B, and 6C depict example dependence plots for an effect of different ventilation parameters on extubation probability, as predicted by the disclosed system, according to various aspects of the subject technology. FIG. 6A depicts a plot 60 which relates to the effect of dynamic compliance. FIG. 6B relates to the effect of peak airway pressure. FIG. 6C relates to the effect of ventilator WOB.

The subject technology discerns which clinical data or features most affect extubation predictions while also delivering information on how these features affect extubation predictions. For example, in some implementations, a predictive model generated according to the subject technology may be operable to predict how a change in dynamic compliance, or ventilator work of breathing, over a range of values will affect the predicted probability of extubation readiness for a specific patient. This may be accomplished, in part, through delivery of partial dependence plots of the most clinically relevant features to the clinician or caretaker. These plots demonstrate the change in predicted extubation probability by sweeping the value of one variable/feature. In the depicted graphs of FIGS. 6A, 6B, and 6C, the y-axis can be interpreted as the change in the predicted probability from the baseline (left-most) feature value. The x-axis can be interpreted as the range of values for the specific feature being analyzed.

FIGS. 6A, 6B, and 6C demonstrate how each specific ventilation feature may have a different effect on predicted extubation probability, depending on its value. For example, as shown in the example of FIG. 6B, an increase in peak airway pressure above 12 cmH20 for a specified patient may lead to a significant decrease in the probability that the patient is ready for extubation. Alternatively, as shown in the example of FIG. 6A, an increase in dynamic compliance for a specified patient past 25 L/cmH20 and up to 50 L/cmH20 greatly increases the probability that a patient is considered an extubation candidate. Creation and delivery of such insights by the subject technology provides physicians or other clinicians useful input to tailor their care for individual patients. Moreover, by considering how these clinical features affect extubation probabilities for each patient differently, the subject technology enables a data-driven objective approach to deciphering the optimal extubation time.

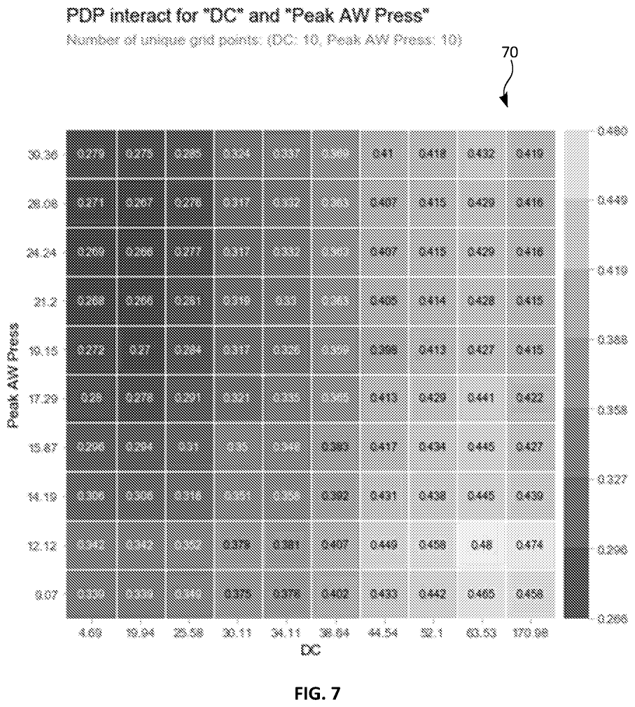

FIG. 7 depicts an example two-dimensional partial dependence plot 70 illustrating a correlation between two ventilation parameters with regard to their effect on extubation probability, according to various aspects of the subject technology. In addition to foregoing single variable partial dependence plots of FIG. 6, the system of the subject technology is configured to determine patient-specific feature interactions, such as the example shown in FIG. 7. In this regard, the system may determine how two variables/features interact with each other to either increase or decrease predicted extubation probabilities. The depicted example, provides analytics regarding the interaction between dynamic compliance and peak airway pressure. In the depicted example, a dynamic compliance value greater than 60 L/cmH20 along with a low peak airway pressure of 12 cmH20 leads to the largest increase in predicted extubation probability.

Additionally or in the alternative, specific information generated by the disclosed system's utilization of the predictive algorithm and model(s) may be delivered to remote devices associated with end users (e.g. clinicians, family, etc.), for example, to alert for patient extubation readiness. A message may be sent to a user, and prompt the user to take action and begin the extubation procedure in a timely manner. Earliest possible prediction of extubation readiness and subsequent successful extubation reduces the probability of prolonged stays, Ventilator Associated Events (VAE), Acute Respiratory Distress Syndrome (ARDS), amongst other negative outcomes, including mortality. The system (including, e.g., the predictive algorithm) may additionally deliver patient-specific feature dependencies to end-users. For example, in some embodiments the system may send patient specific summary reports containing information on informative features and feature value descriptive statistics to end users, enabling them to execute personalized or individualized patient care while providing a holistic perspective of the patient condition. A user interface may be provided to the user, and the user may identify the patient within the user interface. The system may then download ventilation and/or physiological data for the patient, and determine the ventilation feature most suitable for resolving the patient towards extubation.

The system may also include one or more feedback mechanisms that are actuated upon a positive or negative extubation label prediction. In some implementations, when a positive extubation label is identified (thereby predicting a patient's candidacy for extubation), the system may prompt the user to confirm an extubation should be initiated. In response to receiving a confirmation from a user device, the system may cause ventilator settings (of a ventilator associated with the patient) to adjust in preparation for a successful extubation. Additionally or in the alternative, a confirmation of a successful extubation prediction may be fed back into the supervised learning algorithm for real-time re-training for the purposes of continual improvement and learning. This may lead to increased prediction accuracies the longer the algorithm has been in deployment.

Additionally or in the alternative, the predictive algorithm of the subject technology may be used in conjunction with a secondary algorithm, such as a trained Fitted Q-iteration algorithm. The Fitted Q-iteration algorithm may further train the given model to cause the system to deliver an automated and tailored weaning and extubation procedure for the patient based on a given patient state. Using a series of predetermined features (e.g., sampled ventilation parameters), the given model may be trained to deliver a personalized protocol of sedation dosages and ventilator support for the patient. In some embodiments, the feature space for the Fitted Q-iteration algorithm may be a 29-dimensional space, for example, including 19 time-series ventilator features used to predict extubation, plus 10 additional features. These additional features may include, for example, blood gas data, oximetry data, EtCO2 data, current sedative, analgesic or other drug dosages, fixed demographic features of each patient (age, weight, gender, admit type, ethnicity), and level of consciousness (RASS scale). These sampled features may define the current state for a given patient throughout the execution of the predictive algorithm on the model, and become key predictors in weaning protocols and predicting whether the patient is a candidate for extubation.

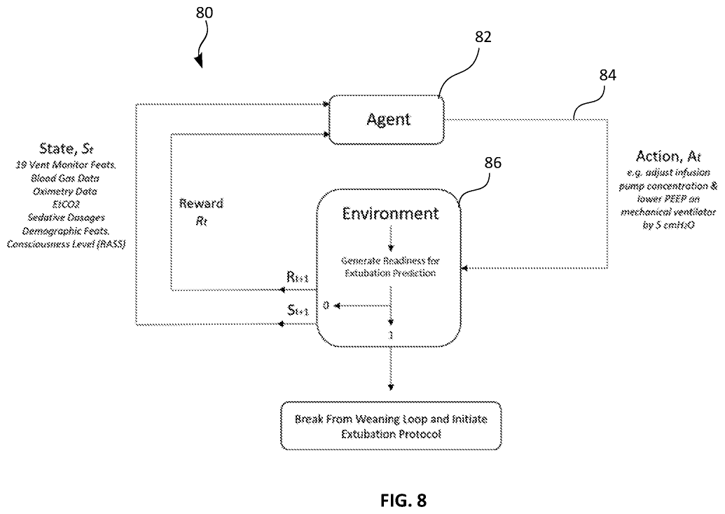

FIG. 8 depicts an example closed-loop system incorporating an automated weaning and extubation model, according to various aspects of the subject technology. The system 80 of the subject technology may be configured to control an agent 82 (e.g., a ventilator and/or infusion device) based on a closed-loop predictive algorithm which updates the given model based on feedback data 84 received from the agent 82. In this manner, the predictive model includes reinforcement learning to facilitate weaning of a patient from ventilator support so that the patient can be timely extubated.

In the depicted example, portions of system 80, including the predictive algorithm and related model(s), is represented as a predictive environment 86. In some implementations, the predictive algorithm may include a fitted Q-iteration algorithm. Actions are iteratively taken by the agent 82, and an extubation readiness prediction is generated by the predictive environment 86 based on the predictive model after every action, or after a given number of actions have been performed. The iterative actions may take place as part of an overall weaning stage implemented by the system.

The feedback loop implemented by predictive environment 86 may continue indefinitely until a positive prediction is generated, or may continue for a predetermined amount of time or number of iterations before requiring user intervention (e.g., to confirm continued operation). Once a positive prediction event occurs (defined by, e.g., a threshold likelihood that the patient is a candidate for extubation), the algorithm breaks out of the loop to begin a predetermined extubation protocol or procedure. This extubation protocol or procedure may include, for example, causing the system to send or initiate messages or notifications to caregivers.

In some implementations, all or most of the features utilized by a given model may be retrieved automatically from a centralized storage location (e.g., data storage 156). In some implementations, some or all features may be updated by the predictive environment 86 at each new state or after every action the agent executes. Alternatively, the system may automatically dispatch one or more requests to an external system for feature updates, and subsequently update the given model with the updated features once received from the external system. For example, the model may automatically order blood gases and/or a consciousness test to be performed based on pre-determined patient-specific conditions. Update requests may occur periodically according to a predetermined schedule. In some implementations, the frequency at which requests are made may be set by a user. Results of these tests are then inputted into the model, updating current state blood gas and consciousness level values. The updated values may then remain static until the next request is dispatched. The value(s) of a previous test result may remain static until a next result is received and overwrites the value(s). In some implementations, the values may be further updated based on other dynamic parameters received by the system.

According to various implementations, when training a model, the predictive environment 86 may map an optimal action to every given patient state, in time, with the end goal of maximizing a long-term reward. For example, when a model is trained, the algorithm (e.g., implemented by system 80 and/or agent 82) may (e.g., semi-randomly) navigate the feature space and assign a numeric value, or reward, to every feature state depending on whether or not that specific state led to a positive extubation prediction in the long term. According to some implementations, good states that are part of a long or short path that ultimately lead to a positive prediction have a higher value associated to it than one that doesn't. In this manner, one or more rewards Rt may be assigned to each possible state.

Rewards for each state are shaped during training based off the end goal of reaching a positive extubation readiness prediction. Once the model is deployed and the system is navigating the feature space, it may do so in a manner in which it maximizes this accumulation of values from state to state until it reaches the end goal of achieving a positive extubation prediction. In this regard, when the reinforcement learning model is trained, all state values/rewards are initialized to zero. Then, the system may first make its way through the feature space (e.g., vent and sedation values) randomly, while generating extubation predictions after every state change (e.g., since there is no optimal policy that has been learned yet). While randomly navigating, if a positive extubation prediction is generated after a sequence of state changes, then all states that were chosen during that time may be positively rewarded with an increase in value. If the algorithm does not receive a positive extubation prediction after a determined number of iterations then that sequence of states may be penalized with a decrease in value. At the end of training, the feature space may be visualized as a high dimensional grid corresponding to all the states and each state (e.g., space on the grid) with its corresponding value. Some state values may be negative (e.g., un-desirable), while others may be positive (desirable). In this regard, once the model is deployed, the system may use this trained grid of state values that has been shaped by the extubation predictions in order to safely change certain vent and infusion pump parameters with the end goal of maximizing its accumulated reward (sum of every state it encounters), which will in turn lead to a positive extubation prediction given the way it was trained. The foregoing encourages actions by the agent 82 that ultimately lead to a positive extubation readiness prediction by safely navigating the feature space to a position where vitals (e.g. FiO2, SpO2, PEEP, etc.) are within a tolerance considered safe to extubate the patient.

Predictive environment 86 may determine an action space which includes, for example, all possible actions that can be taken by the agent while in a state to get to a new state. The action space may include the automatic implementation of discretized changes in levels of sedation or other medications (e.g. delivery rate/dose) and discretized changes in ventilator parameters (e.g. FiO2, PEEP). In this regard, predictive environment 86 may generate a new set of state parameters St, which may then be sent to agent 82, which may execute these parameters directly on a ventilator or infusion device directly. In some implementations, the algorithm may send confirmatory messages or notifications to clinicians or users of settings changes as well as progress indicators indicative of how the patient is progressing towards extubation readiness.

The foregoing closed-loop predictive process may be implemented in tangent with an automated extubation regime that follows after a positive extubation prediction. This automated extubation protocol takes into account the severity of each patient's situation by using the probability of reintubation in order to implement a suitable extubation procedure geared towards each patient's unique risk circumstance.

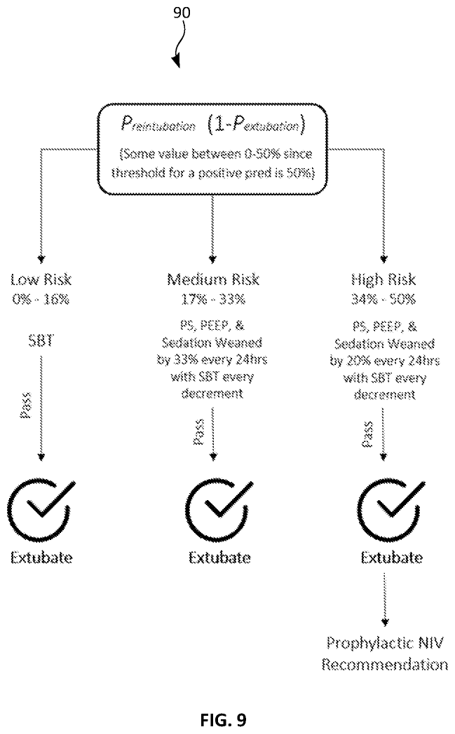

FIG. 9 depicts a flow diagram 90 of an example extubation protocol tailored based on patient reintubation risk, according to various aspects of the subject technology. A positive prediction in the extubation readiness predictive model is output when the probability of a successful extubation is above a certain threshold value (e.g. 0.5). Therefore, the probability of reintubation (unsuccessful extubation) may be represented as 1−Pextubation. This value may only take on values between 0 and the classification threshold value chosen (e.g. 0.5). In some implementations, the predictive model clusters patients into distinct categories based on their severity levels/probabilities of reintubation. For example, in one implementation, patients may be split into three clusters, those with a Preintubation between 0-16%, those with a Preintubation between 17%-33%, and those with a Preintubation between 34%-50%. By choosing the extubation procedure most suitable for the severity of the patient depending on the probability of reintubation, this decision tree approach allows for an automated model to better align care with the severity and risk of the patient's unique circumstance. Depending on the group a specific patient finds themselves in, the ventilator may be programmed to automatically wean the current parameters by adjusting them to correspond to the settings specified for that group, while automatically administering one or more spontaneous breathing trial (SBTs) throughout. It has been found that differentiating the method of extubation and post-extubation plan depending on the severity of the patient greatly reduces the likelihood of complications occurring further down the line. Needs and optimal plan of action for each patient are different depending on that risk of reintubation. In the depicted example, the range is split into three severity levels, however, the number of clusters may be greater or less.

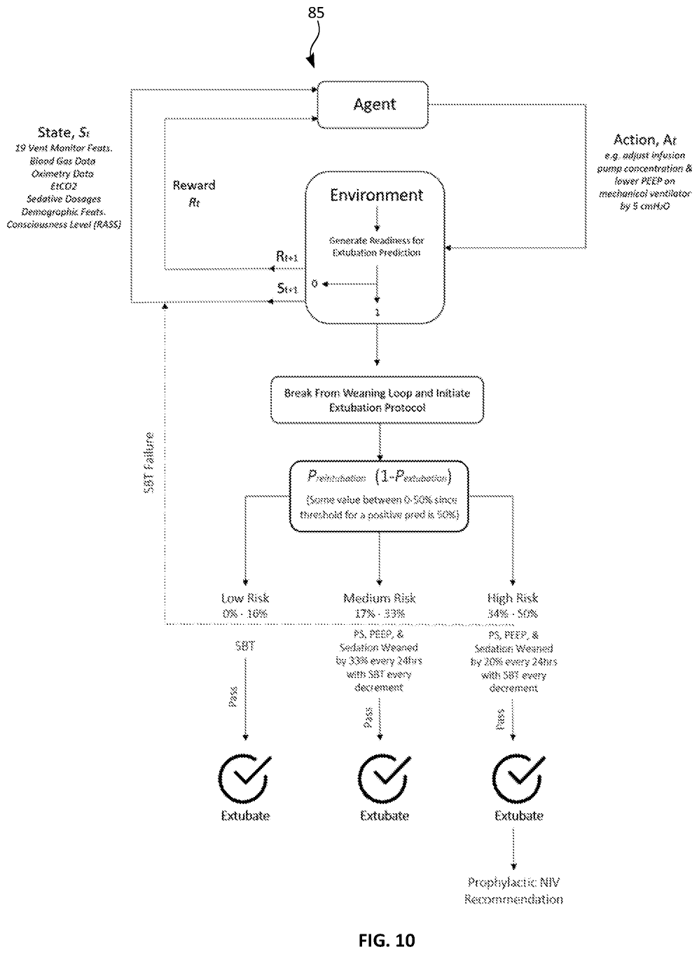

FIG. 10 depicts an example closed-loop system 85 incorporating an automated weaning and extubation model, tailored based on patient reintubation risk, according to various aspects of the subject technology. The severity level associated with each cluster of patients dictates the approach taken to completely wean a particular patient off of the ventilator for extubation and the care that is provided after extubation. As the severity of the cluster increases, the slower the ventilator and infusion pump settings are automatically changed toward weaning the patient from ventilation. For example, those patients in the least severe of clusters may be prompted with a confirmative spontaneous breathing trial (SBT) upon a positive extubation prediction and, if passed, can be subsequently taken off the ventilator. Patients in the most severe of clusters will have pressure support (PS), PEEP, and sedative or other medication dosages weaned gradually (e.g. 20% every 24 hours) with intermittent SBTs automatically triggered after every automatic adjustment. If at any time a patient fails a SBT, the model may loop back into and return to the automatic weaning cycle taken care of by the reinforcement learning algorithm discussed previously.

The foregoing weaning process continues until the patient has successfully passed all SBTs, and PS and sedative or other medication dosage have been completely weaned, and the patient is extubated and completely off the ventilator. In some implementations, the speed in which the patient is weaned differs between clusters and follows a gradient reflecting severity as you move across clusters—i.e. the rate of weaning of sedation and ventilatory support is inversely proportional to the probability of reintubation. In some implementations, prophylactic non-invasive ventilation (NIV) is beneficial in more severe cases and, in these implementations, an automatic recommendation or automated order for prophylactic NIV may be issued by the system for patients in severe clusters or those having greater probability of reintubation.

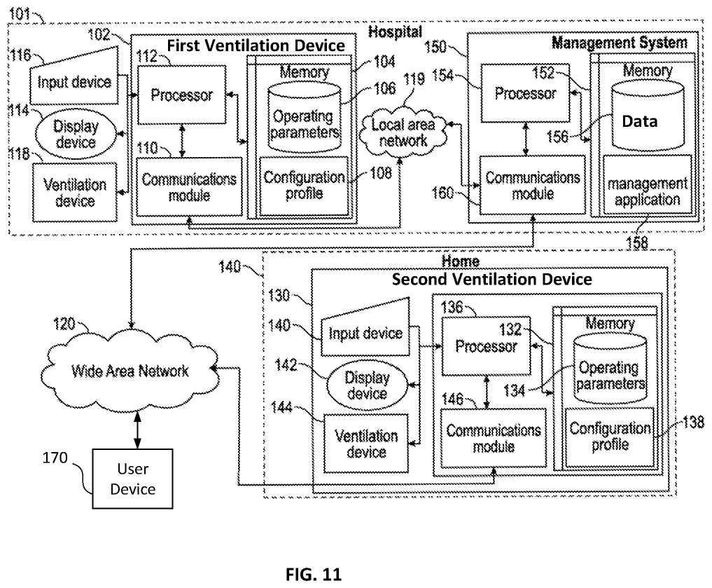

FIG. 11 is a block diagram illustrating an example system for predictive weaning of ventilated patients, including multiple ventilation devices and a ventilation management system, according to certain aspects of the subject technology. The system may assess conditions of ventilated patients and adjusting an operation mode of a ventilator, including ventilation devices 102 and 130, and a management system 150, according to certain aspects of the subject technology. Management system 150 may include a server and, in many aspects, includes logic and instructions for providing the functionality previously described with regard to FIGS. 1 through 10. For example, a server of management system 150 may implement predictive environment 86, including the foregoing predictive algorithm(s) and predictive model(s).

The server of management system 150 may broker communications between the various devices, and/or generate user interface 10 for display by user devices 170. Ventilation device 102 and ventilation device 130 may represent each of multiple ventilation devices connected to management system 150. Although the management system 150 is illustrated as connected to a ventilation device 102 and a ventilation device 130, the management system 150 is configured to also connect to different medical devices, including infusion pumps, point of care vital signs monitors, and pulmonary diagnostics devices. In this regard, device 102 or device 130 may be representative of a different medical device.

Ventilation device 102 is connected to the management system 150 over the LAN 119 via respective communications modules 110 and 160 of the ventilation system 102 and the management system 150. The management system 150 is connected over WAN 120 to the ventilation device 130 via respective communications modules 160 and 146 of the management system 150 and the ventilation device 130. The ventilation device 130 is configured to operate substantially similar to the ventilation device 102 of a hospital system 101, except that the ventilation device (or medical device) 130 is configured for use in the home 140. The communications modules 110, 160, and 146 are configured to interface with the networks to send and receive information, such as data, requests, responses, and commands to other devices on the networks. The communications modules 110, 160, and 146 can be, for example, modems, Ethernet cards, or WiFi component modules and devices.

The management system 150 includes a processor 154, the communications module 160, and a memory 152 that includes hospital data 156 and a management application 158. Although one ventilation device 102 is shown in FIG. 16, the management system 150 is configured to connect with and manage many ventilation devices 102, both ventilation devices 102 for hospitals and corresponding systems 101 and ventilation devices 130 for use in the home 140.

In certain aspects, the management system 150 is configured to manage many ventilation devices 102 in the hospital system 101 according to certain rules and procedures. For example, when powering on, a ventilation system 102 may send a handshake message to the management system 150 to establish a connection with the management system 150. Similarly, when powering down, the ventilation system 102 may send a power down message to the management system 150 so that the management system 150 ceases communication attempts with the ventilation system 102.

The management system 150 is configured to support a plurality of simultaneous connections to different ventilation devices 102 and ventilation devices 130, and to manage message distribution among the different devices, including to and from a user device 170. User device 170 may be a mobile device such as a laptop computer, tablet computer, or mobile phone. User device 170 may also be a desktop or terminal device authorized for use by a user. In this regard, user device 170 is configured with the previously described messaging application depicted by FIGS. 1 through 15 to receive messages, notifications, and other information from management system 150, as described throughout this disclosure.

The number of simultaneous connections can be configured by an administrator in order to accommodate network communication limitations (e.g., limited bandwidth availability). After the ventilation device 102 successfully handshakes with (e.g., connects to) the management system 150, the management system 150 may initiate communications to the ventilation device 102 when information becomes available, or at established intervals. The established intervals can be configured by a user so as to ensure that the ventilation device 102 does not exceed an established interval for communicating with the management system 150.

The management system 150 can receive or provide data to the ventilation device 102, for example, to adjust patient care parameters of the ventilation device. For instance, alerts may be received from ventilation device 102 (or device 130) responsive to thresholds being exceeded. An admit-discharge-transfer communication can be sent to specified ventilation devices 102 within a certain care area of a hospital 101. Orders specific to a patient may be sent to a ventilation device 102 associated with the patient, and data specific to a patient may be received from ventilation device 102.

The ventilation device 102 may initiate a communication to the management system 150 if an alarm occurs on the ventilation system 102. The alarm may be indicated as time-sensitive and sent to the beginning of the queue for communicating data to the management system 150. All other data of the ventilation device 102 may be sent together at once, or a subset of the data can be sent at certain intervals.

Hospital data 156 may continuously or periodically received (in real time or near real time) by management system 150 from each ventilator device 102 and each ventilator device 130. The hospital data 156 may include configuration profiles configured to designate operating parameters for a respective ventilation device 102, operating parameters of each ventilation device 102 and/or physiological statistics of a patient associated with the ventilation device 102. Hospital data 156 also includes patient data for patients admitted to a hospital or within a corresponding hospital system 101, order (e.g., medication orders, respiratory therapy orders) data for patients registered with the hospital 101 system, and/or user data (e.g., for caregivers associated with the hospital system 101). As described previously with regard to the systems described with regard to FIGS. 1 through 10, ventilation parameters may be updated or changed based on an updated state provided by these systems. The parameters may be stored and/or updated in data storage 152.

The physiological statistics and/or measurements of the ventilator data includes, for example, a statistic(s) or measurement(s) indicating compliance of the lung (Cdyn, Cstat), flow resistance of the patient airways (Raw), inspiratory-expiratory ratio (I/E), spontaneous ventilation rate, exhaled tidal volume (Vte), total lung ventilation per minute (Ve), peak expiratory flow rate (PEFR), peak inspiratory flow rate (PIFR), mean airway pressure, peak airway pressure, an average end-tidal expired CO2 and total ventilation rate. The operating parameters include, for example, a ventilation mode, a set mandatory tidal volume, positive-end expiratory pressure (PEEP), an apnea interval, a bias flow, a breathing circuit compressible volume, a patient airway type (for example endotracheal tube, tracheostomy tube, face mask) and size, a fraction of inspired oxygen (FiO2), a breath cycle threshold, and a breath trigger threshold.

The processor 154 of the management system 150 is configured to execute instructions, such as instructions physically coded into the processor 154, instructions received from software (e.g., management application 158) in memory 152, or a combination of both. For example, the processor 154 of the management system 150 executes instructions to receive ventilator data from the ventilation device(s) 102 (e.g., including an initial configuration profile for the ventilation system 102).

Ventilation device 102 is configured to send ventilator information, notifications (or “alarms”), scalars, operating parameters 106 (or “settings”), physiological statistics (or “monitors”) of a patient associated with the ventilation device 102, and general information. The notifications include operational conditions of the ventilation device 102 that may require operator review and corrective action. Scalars include parameters that are typically updated periodically (e.g., every 500 ms) and can be represented graphically on a two-dimensional scale. The physiological statistics represent information that the ventilation device 102 is monitoring, and can be dynamic based on a specific parameter. The operating parameters 106 represent the operational control values that the caregiver has accepted for the ventilation device 102. The general information can be information that is unique to the ventilation device 102, or that may relate to the patient (e.g., a patient identifier). The general information can include an identifier of the version and model of the ventilation device 102. It is also understood that the same or similar data may be communicated between management system 150 and ventilation device 130.

With further reference to FIG. 11, management system 150 may include (among other equipment) a centralized server and at least one data source (e.g., a database 152). The centralized server and data source(s) may include multiple computing devices distributed over a local 119 or wide area network 120, or may be combined in a single device. Data may be stored in data source(s) 152 (e.g., a database) in real time and managed by the centralized server. In this regard, multiple medical devices 102, 130 may communicate patient data, over network 119, 120, to the centralized server in real time as the data is collected or measured from the patient, and the centralized server may store the patient data in data source(s) 152. According to some implementations, one or more servers may receive and store the patient data in multiple data sources.

According to various implementations, management system 150 (including centralized server) is configured to (by way of instructions) generate and provide virtual user interface 10 to clinician devices 170. In some implementations, management system 150 may function as a web server, and virtual interface 100 may rendered from a website provided by management system 150. According to various implementations, management system 150 may aggregate real time patient data and provide the data for display in virtual interface 100. The data and/or virtual interface 100 may be provided (e.g., transmitted) to each clinician device 170, and each clinician device 170 may include a software client program or other instructions configured to, when executed by one or more processors of the device, render and display virtual interface 100 with the corresponding data. The depicted clinician devices 170 may include a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity. While not shown in FIG. 16, it is understood that the connections between the various devices over local network 119 or wide area network 120 may be made via a wireless connection such as WiFi, BLUETOOTH, Radio Frequency, cellular, or other similar connection.

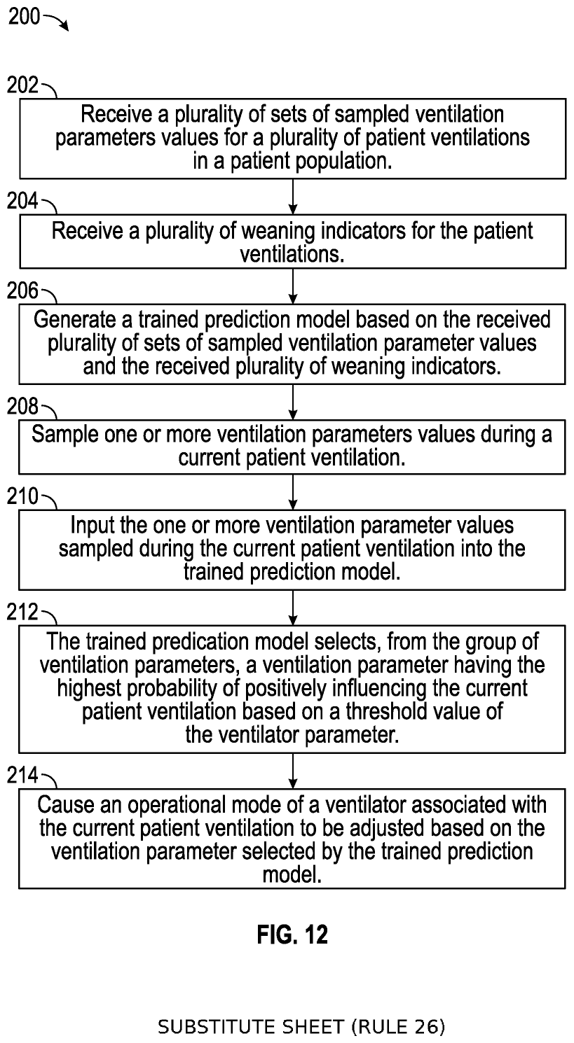

FIG. 12 depicts an example flow chart of a method of generating patient-specific ventilation settings based on a trained prediction model, and for adjusting an operation of a ventilator to wean a patient from ventilation, according to aspects of the subject technology. The process 200 is implemented, in part, through the exchange of data between the ventilation device 102 (or device 130), the management system 150, and user device 170. For explanatory purposes, the various blocks of example process 500 are described herein with reference to FIGS. 1 through 11, and the components and/or processes described herein. The one or more of the blocks of process 200 may be implemented, for example, by a computing device, including a processor and other components utilized by the device. In some implementations, one or more of the blocks may be implemented apart from other blocks, and by one or more different processors or devices. Further for explanatory purposes, the blocks of example process 200 are described as occurring in serial, or linearly. However, multiple blocks of example process 200 may occur in parallel. In addition, the blocks of example process 200 need not be performed in the order shown and/or one or more of the blocks of example process 200 need not be performed.

The example process may be implemented by a system comprising a ventilation communication device configured to receive ventilation data, a medication delivery communication device configured to receive medication delivery information associated with an ongoing administration of a medication to the patient, and one or more sensors configured to obtain physiological data from a patient. The disclosed system may include a memory storing instructions and data, and one or more processors configured to execute the instructions to perform operations.

A trained prediction model is generated based on sets of sampled ventilation parameter values received from the ventilation communication device, and a plurality of weaning indicators representing an outcome of each sampled patient. Ventilation parameter values are sampled during a current patient ventilation and input into the trained prediction model. The model selects, from the group of ventilation parameters, a ventilation parameter and associated parameter value or range of parameter values having the highest probability of positively influencing the current patient ventilation based on a threshold value of the ventilator parameter. The system may then use the returned parameter value(s) to cause an operational mode of a ventilator associated with the current patient ventilation to be adjusted

In the depicted example flow diagram, management system 150 receives a plurality of sets of sampled ventilation parameter values for a plurality of patient ventilations in a patient population (202). Each patient ventilation is associated with a group of ventilation parameters for which one or more of the plurality of sets is sampled during a same time period. The sampled parameters may be received by management system 150 via a ventilation communication device (e.g., in the form of a communication module 110 or 160), which may be configured to receive the individual parameters and organize them into the sets. According to various aspects, each of the sets of sampled ventilation parameter values corresponds to a ventilation statistic or measurement indicating one of compliance of the lung (Cdyn, Cstat), flow resistance of the patient airways (Raw), inspiratory-expiratory ratio (I/E), spontaneous ventilation rate, exhaled tidal volume (Vte), total lung ventilation per minute (Ve), peak expiratory flow rate (PEFR), peak inspiratory flow rate (PIFR), mean airway pressure, peak airway pressure, an average end-tidal expired CO2, total ventilation rate, a set mandatory tidal volume, positive end expiratory pressure (PEEP), an apnea interval, a bias flow, a breathing circuit compressible volume, a patient airway type or size, a fraction of inspired oxygen (FiO2), a breath cycle threshold, or a breath trigger threshold.

In some implementations, system 101 includes a medication delivery communication device (e.g., in the form of a communication module 110 or 160) configured to receive current medication delivery information associated with an ongoing administration of a medication to the patient. In these implementations, medication delivery information is received by management system 150 from the medication delivery communication device. Medication delivery information may include, for example, a flow rate of a medication, a bolus amount, an amount of the medication administered over a period of time, and the like. According to some implementations, diagnostic information is received for a patient ventilation by the management system 150, and the management system 150 may determine, based on signals received from the one or more sensors, a physiological state of the patient.

Management system 150 also receives a plurality of weaning indicators (204). Each of the plurality of weaning indicators corresponds to a respective patient ventilation of the plurality of patient ventilations and the one or more of the plurality of sets sampled during the same time period associated with the respective patient ventilation. Each of the weaning indicators may correspond to a patient outcome for a respective patient of the plurality of patients, with each patient outcome indicating whether the patient ventilation associated with a given patient was reduced or terminated during the given sampling period. Additionally or in the alternative, each weaning indicator may indicate whether a respective patient was extubated during the same time period associated with the respective patient ventilation.

A trained prediction model is generated based on the received plurality of sets of sampled ventilation parameter values and the received plurality of weaning indicators (206). The trained prediction model is trained to select, based on an input of ventilation parameter values for a patient ventilation, one or more ventilator parameters having a highest probability, within the group of ventilation parameters, of positively influencing the patient ventilation based on respective threshold values.

During a current patient ventilation, one or more ventilation parameter values are sampled and received by the system 150 during the current patient ventilation (208). Management system 150 automatically inputs the one or more ventilation parameter values sampled during the current patient ventilation into the trained prediction model (210).

Based on the inputting of the plurality of ventilation parameters, the trained prediction model selects, from the group of ventilation parameters, a ventilation parameter having the highest probability of positively influencing the current patient ventilation based on a threshold value of the ventilator parameter (212). According to various aspects, the trained prediction model selects, with the selected ventilation parameter, a parameter value or range of parameter values for the selected ventilation parameter and which satisfies the threshold value of the ventilator parameter. For example, the model may determine a likelihood that a patient associated with the current patient ventilation is a candidate for extubation and termination of the current ventilation based on an adjustment of a current operational mode of a ventilator 102, 130 based on the ventilation parameter being set to the parameter value or to a value within the range of parameter values. The selection process may be based on this determination.

Management system 150 may then cause the operational mode of the ventilator 102, 130 associated with the current patient ventilation to be adjusted based on the ventilation parameter and/or value(s) selected by the trained prediction model (214). Additionally or in the alternative, management system 150 may send the selected ventilation parameter and the parameter value or range of parameter values to a computing device associated with a clinician assigned to the current patient ventilation.

As described previously, the foregoing process may be part of a closed-loop operational cycle. In this regard, management system 150 may set the selected ventilation parameter on the ventilator with the parameter value or range of parameter values received from the trained prediction model. A plurality of updated ventilation parameter values sampled during the current patient ventilation may be received after setting the selected ventilation parameter. System 150 may automatically input the plurality of updated ventilation parameter values sampled during the current patient ventilation into the trained prediction model. Based on this input, system 150 may then receive, from the trained prediction model, an updated ventilation parameter selected from the group of ventilation parameters and an updated parameter value or updated range of parameter values for the updated ventilation parameter, and then set the updated ventilation parameter on the ventilator with the updated parameter value or updated value within the updated range of parameter values received from the trained prediction model.

According to some implementations, the trained prediction model may assign the current patient ventilation to one of a plurality of cluster categories based on the plurality of ventilation parameter values sampled during the current patient ventilation. In this regard, each cluster category may be associated with a probability that current patient ventilation is a candidate for extubation or termination of the current ventilation. The trained prediction model may then select the updated parameter value or updated value within the updated range of parameter values based on the cluster category assigned to the current patient ventilation. As the probability decreases, the less the updated parameter value or updated value within the updated range of parameter values differs from a current value of the updated parameter value toward weaning or terminating the current ventilation.

According to some implementations, the foregoing modeling, calculations and/or determinations may be facilitated, at least in part, by a neural network. For example, system 150 may provide the sampled ventilation parameters, physiological parameters of the patient, a determined physical state of the patient, the determined operational mode of the ventilator, the medication delivery information, other diagnostic information for the patient to the neural network, and receives, from the neural network, the selected weaning parameter value(s) or range of values. The neural network may further be used to correlate the received data and/or the generated models with candidate results to determine optimal ventilation parameters. The system 150 then adjusts, based on the determined optimal ventilation parameters, one or more current operating parameters of the ventilator 102, 130, wherein adjusting the parameter may influence the operational mode of the ventilator, as described previously.

According to various implementations, physiogical data may be received from one or more sensors. The sensors may include a sensor configured to obtain a vital sign measurement of the patient, including one or more of blood pressure, patient core temperature, heart rate, electrocardiogram (ECG) signal, pulse, or blood oxygen saturation level, wherein the determined physiological state of the patient comprises information representative of the vital sign measurement. The sensors may include a sensor applied to the patient's skin and configured to measure a level of muscle tension. In some implementations, the medication delivery communication device (e.g., component 14) is configured to receive, from an infusion pump, the medication delivery information, the medication delivery information comprising a drug identification, drug concentration, drug dosage, or length of an ongoing infusion. In some implementations, management system 150 (or hospital system 101) is configured to receive diagnostic information for the patient. The diagnostic information may include lab results associated with the patient received from a diagnostic information system.

Many aspects of the above-described example 200, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RANI chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

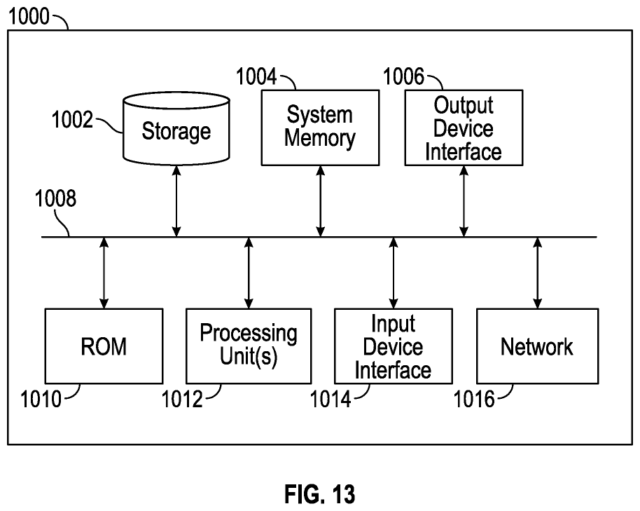

FIG. 13 is a conceptual diagram illustrating an example electronic system for generating patient-specific ventilation settings based on a trained prediction model, and for adjusting an operation of a ventilator, according to aspects of the subject technology. Electronic system 1000 may be a computing device for execution of software associated with one or more portions or steps of process 1000, or components and processes provided by FIGS. 1 through 12. Electronic system 1000 may be representative, in combination with the disclosure regarding FIGS. 1 through 9, of the management system 150 (or server of system 150) or the clinician device(s) 170 described above. In this regard, electronic system 1000 or computing device may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 1000 may include various types of computer readable media and interfaces for various other types of computer readable media. In the depicted example, electronic system 1700 includes a bus 1008, processing unit(s) 1012, a system memory 1004, a read-only memory (ROM) 1010, a permanent storage device 1002, an input device interface 1014, an output device interface 1006, and one or more network interfaces 1016. In some implementations, electronic system 1000 may include or be integrated with other computing devices or circuitry for operation of the various components and processes previously described.

Bus 1008 collectively represents all system, peripheral, and chipset buses that communicatively connect the numerous internal devices of electronic system 1000. For instance, bus 1008 communicatively connects processing unit(s) 1012 with ROM 1010, system memory 1004, and permanent storage device 1002.

From these various memory units, processing unit(s) 1012 retrieves instructions to execute and data to process in order to execute the processes of the subject disclosure. The processing unit(s) can be a single processor or a multi-core processor in different implementations.

ROM 1010 stores static data and instructions that are needed by processing unit(s) 1012 and other modules of the electronic system. Permanent storage device 1002, on the other hand, is a read-and-write memory device. This device is a non-volatile memory unit that stores instructions and data even when electronic system 1000 is off. Some implementations of the subject disclosure use a mass-storage device (such as a magnetic or optical disk and its corresponding disk drive) as permanent storage device 1002.

Other implementations use a removable storage device (such as a floppy disk, flash drive, and its corresponding disk drive) as permanent storage device 1002. Like permanent storage device 1002, system memory 1004 is a read-and-write memory device. However, unlike storage device 1002, system memory 1004 is a volatile read-and-write memory, such a random access memory. System memory 1004 stores some of the instructions and data that the processor needs at runtime. In some implementations, the processes of the subject disclosure are stored in system memory 1004, permanent storage device 1002, and/or ROM 1010. From these various memory units, processing unit(s) 1012 retrieves instructions to execute and data to process in order to execute the processes of some implementations.

Bus 1008 also connects to input and output device interfaces 1014 and 1006. Input device interface 1014 enables the user to communicate information and select commands to the electronic system. Input devices used with input device interface 1014 include, e.g., alphanumeric keyboards and pointing devices (also called “cursor control devices”). Output device interfaces 1006 enables, e.g., the display of images generated by the electronic system 1000. Output devices used with output device interface 1006 include, e.g., printers and display devices, such as cathode ray tubes (CRT) or liquid crystal displays (LCD). Some implementations include devices such as a touchscreen that functions as both input and output devices.

Also, as shown in FIG. 10, bus 1008 also couples electronic system 1700 to a network (not shown) through network interfaces 1016. Network interfaces 1016 may include, e.g., a wireless access point (e.g., Bluetooth or WiFi) or radio circuitry for connecting to a wireless access point. Network interfaces 1016 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 1700 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML, page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A machine-implemented method for assessing a condition of a ventilated patient and adjusting an operation mode of the ventilator, comprising: receiving, by one or more computing devices, a plurality of sets of sampled ventilation parameter values for a plurality of patient ventilations in a patient population, each patient ventilation being associated with a group of ventilation parameters for which one or more of the plurality of sets is sampled during a same time period; receiving, by the one or more computing devices, a plurality of weaning indicators, each of the plurality of weaning indicators corresponding to a respective patient ventilation of the plurality of patient ventilations and the one or more of the plurality of sets sampled during the same time period associated with the respective patient ventilation; generating, by the one or more computing devices, a trained prediction model based on the received plurality of sets of sampled ventilation parameter values and the received plurality of weaning indicators, the trained prediction model being trained to select, based on an input of ventilation parameter values for a patient ventilation, one or more ventilator parameters having a highest probability, within the group of ventilation parameters, of positively influencing the patient ventilation based on respective threshold values; receiving, by the one or more computing devices, a plurality of ventilation parameter values sampled during a current patient ventilation; automatically inputting, by the one or more computing devices, the plurality of ventilation parameter values sampled during the current patient ventilation into the trained prediction model; receiving, from the trained prediction model, by the one or more computing devices, based on the inputting of the plurality of ventilation parameters, a ventilation parameter selected from the group of ventilation parameters and having the highest probability of positively influencing the current patient ventilation based on a threshold value of the ventilator parameter; and causing, by the one or more computing devices, an operational mode of a ventilator associated with the current patient ventilation to be adjusted based on the ventilation parameter selected by the trained prediction model.

Clause 2. The method of Clause 1, further comprising: receiving, from the trained prediction model, with the selected ventilation parameter, a parameter value or range of parameter values for the selected ventilation parameter and which satisfies the threshold value of the ventilator parameter.

Clause 3. The method of Clause 2, further comprising: setting the selected ventilation parameter on the ventilator with the parameter value or range of parameter values received from the trained prediction model; receiving a plurality of updated ventilation parameter values sampled during the current patient ventilation, after setting the selected ventilation parameter; automatically inputting the plurality of updated ventilation parameter values sampled during the current patient ventilation into the trained prediction model; receiving, from the trained prediction model, based on the inputting of the plurality of updated ventilation parameters, an updated ventilation parameter selected from the group of ventilation parameters and an updated parameter value or updated range of parameter values for the updated ventilation parameter; and setting the updated ventilation parameter on the ventilator with the updated parameter value or updated value within the updated range of parameter values received from the trained prediction model.

Clause 4. The method of Clause 3, further comprising: by the trained prediction model, assigning the current patient ventilation to one of a plurality of cluster categories based on the plurality of ventilation parameter values sampled during the current patient ventilation, each cluster category being associated with a probability that current patient ventilation is a candidate for extubation or termination of the current ventilation; and by the trained prediction model, selecting the updated parameter value or updated value within the updated range of parameter values based on the cluster category assigned to the current patient ventilation, wherein as the probability decreases, the less the updated parameter value or updated value within the updated range of parameter values differs from a current value of the updated parameter value toward weaning or terminating the current ventilation.

Clause 5. The method of Clause 2 or 3, further comprising: determining a likelihood that a patient associated with the current patient ventilation is a candidate for extubation and termination of the current ventilation based on the adjustment of the operational mode of the ventilator based on the ventilation parameter being set to the parameter value or to a value within the range of parameter values.

Clause 6. The method of Clause 5, further comprising: sending the selected ventilation parameter and the parameter value or range of parameter values to a computing device associated with a clinician assigned to the current patient ventilation.

Clause 7. The method of any of the preceding clauses, wherein each of the plurality of weaning indicators indicates whether a respective patient was extubated during the same time period associated with the respective patient ventilation.

Clause 8. The method of any of the preceding clauses, wherein each of the plurality of weaning indicators corresponds to a patient outcome for a respective patient of the plurality of patients, each patient outcome indicating whether the patient ventilation associated with a given patient was reduced or terminated during the sampling period.

Clause 9. The method of any of the preceding clauses, wherein a respective set of the plurality of sets of sampled ventilation parameter values corresponds to a ventilation statistic or measurement indicating one of compliance of the lung (Cdyn, Cstat), flow resistance of the patient airways (Raw), inspiratory-expiratory ratio (FE), spontaneous ventilation rate, exhaled tidal volume (Vte), total lung ventilation per minute (Ve), peak expiratory flow rate (PEFR), peak inspiratory flow rate (PIFR), mean airway pressure, peak airway pressure, an average end-tidal expired CO2, total ventilation rate, a set mandatory tidal volume, positive end expiratory pressure (PEEP), an apnea interval, a bias flow, a breathing circuit compressible volume, a patient airway type or size, a fraction of inspired oxygen (FiO2), a breath cycle threshold, or a breath trigger threshold.

Clause 10. The method of Clause 9, wherein a respective set of the plurality of sets of sampled ventilation parameter values corresponds to a vital sign measurement of the patient indicating one of a blood pressure, patient core temperature, heart rate, electrocardiogram (ECG) signal, pulse, or blood oxygen saturation level.

Clause 11. A system, comprising: a memory storing instructions; and one or more processors configured to execute the instructions to: receive a plurality of sets of sampled ventilation parameter values for a plurality of patient ventilations in a patient population, each patient ventilation being associated with a group of ventilation parameters for which one or more of the plurality of sets is sampled during a same time period; receive a plurality of weaning indicators, each of the plurality of weaning indicators corresponding to a respective patient ventilation of the plurality of patient ventilations and the one or more of the plurality of sets sampled during the same time period associated with the respective patient ventilation; generate a trained prediction model based on the received plurality of sets of sampled ventilation parameter values and the received plurality of weaning indicators, the trained prediction model being trained to select, based on an input of ventilation parameter values for a patient ventilation, one or more ventilator parameters having a highest probability, within the group of ventilation parameters, of positively influencing the patient ventilation based on respective threshold values; receive a plurality of ventilation parameter values sampled during a current patient ventilation; automatically input the plurality of ventilation parameter values sampled during the current patient ventilation into the trained prediction model; receive by the one or more computing devices, based on the inputting of the plurality of ventilation parameters, a ventilation parameter selected from the group of ventilation parameters and having the highest probability of positively influencing the current patient ventilation based on a threshold value of the ventilator parameter; and cause an operational mode of a ventilator associated with the current patient ventilation to be adjusted based on the ventilation parameter selected by the trained prediction model.

Clause 12. The system of Clause 11, wherein the one or more processors are further configured to execute the instructions to: receive, from the trained prediction model, with the selected ventilation parameter, a parameter value or range of parameter values for the selected ventilation parameter and which satisfies the threshold value of the ventilator parameter.

Clause 13. The system of Clause 12, wherein the one or more processors are further configured to execute the instructions to: set the selected ventilation parameter on the ventilator with the parameter value or range of parameter values received from the trained prediction model; receive a plurality of updated ventilation parameter values sampled during the current patient ventilation, after setting the selected ventilation parameter; automatically input the plurality of updated ventilation parameter values sampled during the current patient ventilation into the trained prediction model; receive, from the trained prediction model, based on the inputting of the plurality of updated ventilation parameters, an updated ventilation parameter selected from the group of ventilation parameters and an updated parameter value or updated range of parameter values for the updated ventilation parameter; and set the updated ventilation parameter on the ventilator with the updated parameter value or updated value within the updated range of parameter values received from the trained prediction model.

Clause 14. The system of Clause 13, wherein the one or more processors are further configured to execute the instructions to: cause the trained prediction model to assign the current patient ventilation to one of a plurality of cluster categories based on the plurality of ventilation parameter values sampled during the current patient ventilation, each cluster category being associated with a probability that current patient ventilation is a candidate for extubation or termination of the current ventilation; and cause the trained prediction model to select the updated parameter value or updated value within the updated range of parameter values based on the cluster category assigned to the current patient ventilation, wherein as the probability decreases, the less the updated parameter value or updated value within the updated range of parameter values differs from a current value of the updated parameter value toward weaning or terminating the current ventilation.

Clause 15. The system of any of Clauses 12 through 14, wherein the one or more processors are further configured to execute the instructions to: determine a likelihood that a patient associated with the current patient ventilation is a candidate for extubation and termination of the current ventilation based on the adjustment of the operational mode of the ventilator based on the ventilation parameter being set to the parameter value or to a value within the range of parameter values.

Clause 16. The system of Clause 15, wherein the one or more processors are further configured to execute the instructions to: send the selected ventilation parameter and the parameter value or range of parameter values to a computing device associated with a clinician assigned to the current patient ventilation.

Clause 17. The system of Clauses 11 through 16, wherein each of the plurality of weaning indicators indicates whether a respective patient was extubated during the same time period associated with the respective patient ventilation.

Clause 18. The system of Clauses 11 through 17, wherein each of the plurality of weaning indicators includes or corresponds to a patient outcome for a respective patient of the plurality of patients, each patient outcome indicating whether the patient ventilation associated with a given patient was reduced or terminated during the sampling period.

Clause 19. The system of Clauses 11 through 18, further comprising: a ventilation communication device configured to receive the sampled ventilation parameter values; a medication delivery communication device configured to receive current medication delivery information associated with an ongoing administration of a medication to the patient, wherein the one or more processors are further configured to: execute the instructions to organize the received sampled ventilation parameter values and to organize the sampled ventilation parameter values into the plurality of sets of sampled ventilation parameter values, and automatically input the current medication delivery information into the trained prediction model, wherein the trained prediction model is further trained based on previously known medication delivery information, and wherein the trained prediction model selects the one or more ventilator parameters having the highest probability of positively influencing the patient ventilation based on the respective threshold values and the current medication delivery information.

Claim 20. A non-transitory computer-readable medium comprising instructions, which when executed by a computing device, cause the computing device to perform operations comprising: receiving, by one or more computing devices, a plurality of sets of sampled ventilation parameter values for a plurality of patient ventilations in a patient population, each patient ventilation being associated with a group of ventilation parameters for which one or more of the plurality of sets is sampled during a same time period; receiving, by the one or more computing devices, a plurality of weaning indicators, each of the plurality of weaning indicators corresponding to a respective patient ventilation of the plurality of patient ventilations and the one or more of the plurality of sets sampled during the same time period associated with the respective patient ventilation; generating, by the one or more computing devices, a trained prediction model based on the received plurality of sets of sampled ventilation parameter values and the received plurality of weaning indicators, the trained prediction model being trained to select, based on an input of ventilation parameter values for a patient ventilation, one or more ventilator parameters having a highest probability, within the group of ventilation parameters, of positively influencing the patient ventilation based on respective threshold values; receiving, by the one or more computing devices, a plurality of ventilation parameter values sampled during a current patient ventilation; automatically inputting, by the one or more computing devices, the plurality of ventilation parameter values sampled during the current patient ventilation into the trained prediction model; receiving, from the trained prediction model, by the one or more computing devices, based on the inputting of the plurality of ventilation parameters, a ventilation parameter selected from the group of ventilation parameters and having the highest probability of positively influencing the current patient ventilation based on a threshold value of the ventilator parameter; and causing, by the one or more computing devices, an operational mode of a ventilator associated with the current patient ventilation to be adjusted based on the ventilation parameter selected by the trained prediction model.

Further Consideration:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit this disclosure.

The term website, as used herein, may include any aspect of a website, including one or more web pages, one or more servers used to host or store web related content, etc. Accordingly, the term website may be used interchangeably with the terms web page and server. The predicate words “configured to,” “operable to,” and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A machine-implemented method for assessing a condition of a ventilated patient and adjusting an operation mode of the ventilator, comprising:
receiving, by one or more computing devices, a plurality of sets of sampled ventilation parameter values for a plurality of patient ventilations in a patient population, each patient ventilation being associated with a group of ventilation parameters for which one or more of the plurality of sets is sampled during a same time period;
receiving, by the one or more computing devices, a plurality of weaning indicators, each of the plurality of weaning indicators corresponding to a respective patient ventilation of the plurality of patient ventilations and the one or more of the plurality of sets sampled during the same time period associated with the respective patient ventilation;
generating, by the one or more computing devices, a trained prediction model based on the received plurality of sets of sampled ventilation parameter values and the received plurality of weaning indicators, the trained prediction model being trained to select, based on an input of ventilation parameter values for a patient ventilation, one or more ventilator parameters having a highest probability, within the group of ventilation parameters, of positively influencing the patient ventilation based on respective threshold values;
receiving, by the one or more computing devices, a plurality of ventilation parameter values sampled during a current patient ventilation;
automatically inputting, by the one or more computing devices, the plurality of ventilation parameter values sampled during the current patient ventilation into the trained prediction model;
receiving, from the trained prediction model, by the one or more computing devices, based on the inputting of the plurality of ventilation parameters, a ventilation parameter selected from the group of ventilation parameters and having the highest probability of positively influencing the current patient ventilation based on a threshold value of the ventilator parameter; and
causing, by the one or more computing devices, an operational mode of a ventilator associated with the current patient ventilation to be adjusted based on the ventilation parameter selected by the trained prediction model.

2. The method of claim 1, further comprising:
receiving, from the trained prediction model, with the selected ventilation parameter, a parameter value or range of parameter values for the selected ventilation parameter and which satisfies the threshold value of the ventilator parameter.

3. The method of claim 2, further comprising:
setting the selected ventilation parameter on the ventilator with the parameter value or range of parameter values received from the trained prediction model;
receiving a plurality of updated ventilation parameter values sampled during the current patient ventilation, after setting the selected ventilation parameter;
automatically inputting the plurality of updated ventilation parameter values sampled during the current patient ventilation into the trained prediction model;
receiving, from the trained prediction model, based on the inputting of the plurality of updated ventilation parameters, an updated ventilation parameter selected from the group of ventilation parameters and an updated parameter value or updated range of parameter values for the updated ventilation parameter; and
setting the updated ventilation parameter on the ventilator with the updated parameter value or updated value within the updated range of parameter values received from the trained prediction model.

4. The method of claim 3, further comprising:
by the trained prediction model, assigning the current patient ventilation to one of a plurality of cluster categories based on the plurality of ventilation parameter values sampled during the current patient ventilation, each cluster category being associated with a probability that current patient ventilation is a candidate for extubation or termination of the current ventilation; and
by the trained prediction model, selecting the updated parameter value or updated value within the updated range of parameter values based on the cluster category assigned to the current patient ventilation, wherein as the probability decreases, the less the updated parameter value or updated value within the updated range of parameter values differs from a current value of the updated parameter value toward weaning or terminating the current ventilation.

5. The method of claim 2, further comprising:
determining a likelihood that a patient associated with the current patient ventilation is a candidate for extubation and termination of the current ventilation based on the adjustment of the operational mode of the ventilator based on the ventilation parameter being set to the parameter value or to a value within the range of parameter values.

6. The method of claim 5, further comprising:
sending the selected ventilation parameter and the parameter value or range of parameter values to a computing device associated with a clinician assigned to the current patient ventilation.

7. The method of claim 1, wherein each of the plurality of weaning indicators indicates whether a respective patient was extubated during the same time period associated with the respective patient ventilation.

8. The method of claim 1, wherein each of the plurality of weaning indicators corresponds to a patient outcome for a respective patient of the plurality of patients, each patient outcome indicating whether the patient ventilation associated with a given patient was reduced or terminated during the sampling period.

9. The method of claim 1, wherein a respective set of the plurality of sets of sampled ventilation parameter values corresponds to a ventilation statistic or measurement indicating one of compliance of the lung (Cdyn, Cstat), flow resistance of the patient airways (Raw), inspiratory-expiratory ratio (FE), spontaneous ventilation rate, exhaled tidal volume (Vte), total lung ventilation per minute (Ve), peak expiratory flow rate (PEFR), peak inspiratory flow rate (PIFR), mean airway pressure, peak airway pressure, an average end-tidal expired CO2, total ventilation rate, a set mandatory tidal volume, positive end expiratory pressure (PEEP), an apnea interval, a bias flow, a breathing circuit compressible volume, a patient airway type or size, a fraction of inspired oxygen (FiO2), a breath cycle threshold, or a breath trigger threshold.

10. The method of claim 9, wherein a respective set of the plurality of sets of sampled ventilation parameter values corresponds to a vital sign measurement of the patient indicating one of a blood pressure, patient core temperature, heart rate, electrocardiogram (ECG) signal, pulse, or blood oxygen saturation level.

11. A system, comprising:
a memory storing instructions; and
one or more processors configured to execute the instructions to:
receive a plurality of sets of sampled ventilation parameter values for a plurality of patient ventilations in a patient population, each patient ventilation being associated with a group of ventilation parameters for which one or more of the plurality of sets is sampled during a same time period;
receive a plurality of weaning indicators, each of the plurality of weaning indicators corresponding to a respective patient ventilation of the plurality of patient ventilations and the one or more of the plurality of sets sampled during the same time period associated with the respective patient ventilation;
generate a trained prediction model based on the received plurality of sets of sampled ventilation parameter values and the received plurality of weaning indicators, the trained prediction model being trained to select, based on an input of ventilation parameter values for a patient ventilation, one or more ventilator parameters having a highest probability, within the group of ventilation parameters, of positively influencing the patient ventilation based on respective threshold values;
receive a plurality of ventilation parameter values sampled during a current patient ventilation;
automatically input the plurality of ventilation parameter values sampled during the current patient ventilation into the trained prediction model;
receive by the one or more computing devices, based on the inputting of the plurality of ventilation parameters, a ventilation parameter selected from the group of ventilation parameters and having the highest probability of positively influencing the current patient ventilation based on a threshold value of the ventilator parameter; and
cause an operational mode of a ventilator associated with the current patient ventilation to be adjusted based on the ventilation parameter selected by the trained prediction model.

12. The system of claim 11, wherein the one or more processors are further configured to execute the instructions to:
receive, from the trained prediction model, with the selected ventilation parameter, a parameter value or range of parameter values for the selected ventilation parameter and which satisfies the threshold value of the ventilator parameter.

13. The system of claim 12, wherein the one or more processors are further configured to execute the instructions to:
set the selected ventilation parameter on the ventilator with the parameter value or range of parameter values received from the trained prediction model;
receive a plurality of updated ventilation parameter values sampled during the current patient ventilation, after setting the selected ventilation parameter;
automatically input the plurality of updated ventilation parameter values sampled during the current patient ventilation into the trained prediction model;
receive, from the trained prediction model, based on the inputting of the plurality of updated ventilation parameters, an updated ventilation parameter selected from the group of ventilation parameters and an updated parameter value or updated range of parameter values for the updated ventilation parameter; and
set the updated ventilation parameter on the ventilator with the updated parameter value or updated value within the updated range of parameter values received from the trained prediction model.

14. The system of claim 13, wherein the one or more processors are further configured to execute the instructions to:
cause the trained prediction model to assign the current patient ventilation to one of a plurality of cluster categories based on the plurality of ventilation parameter values sampled during the current patient ventilation, each cluster category being associated with a probability that current patient ventilation is a candidate for extubation or termination of the current ventilation; and
cause the trained prediction model to select the updated parameter value or updated value within the updated range of parameter values based on the cluster category assigned to the current patient ventilation, wherein as the probability decreases, the less the updated parameter value or updated value within the updated range of parameter values differs from a current value of the updated parameter value toward weaning or terminating the current ventilation.

15. The system of claim 12, wherein the one or more processors are further configured to execute the instructions to:
determine a likelihood that a patient associated with the current patient ventilation is a candidate for extubation and termination of the current ventilation based on the adjustment of the operational mode of the ventilator based on the ventilation parameter being set to the parameter value or to a value within the range of parameter values.

16. The system of claim 15, wherein the one or more processors are further configured to execute the instructions to:
send the selected ventilation parameter and the parameter value or range of parameter values to a computing device associated with a clinician assigned to the current patient ventilation.

17. The system of claim 11, wherein each of the plurality of weaning indicators indicates whether a respective patient was extubated during the same time period associated with the respective patient ventilation.

18. The system of claim 11, wherein each of the plurality of weaning indicators corresponds to a patient outcome for a respective patient of the plurality of patients, each patient outcome indicating whether the patient ventilation associated with a given patient was reduced or terminated during the sampling period.

19. The system of claim 11, further comprising:
a ventilation communication device configured to receive the sampled ventilation parameter values;
a medication delivery communication device configured to receive current medication delivery information associated with an ongoing administration of a medication to the patient,
wherein wherein the one or more processors are further configured to:
execute the instructions to organize the received sampled ventilation parameter values and to organize the sampled ventilation parameter values into the plurality of sets of sampled ventilation parameter values, and
automatically input the current medication delivery information into the trained prediction model, wherein the trained prediction model is further trained based on previously known medication delivery information, and
wherein the trained prediction model selects the one or more ventilator parameters having the highest probability of positively influencing the patient ventilation based on the respective threshold values and the current medication delivery information.

20. A non-transitory computer-readable medium comprising instructions, which when executed by a computing device, cause the computing device to perform operations comprising:
receiving, by one or more computing devices, a plurality of sets of sampled ventilation parameter values for a plurality of patient ventilations in a patient population, each patient ventilation being associated with a group of ventilation parameters for which one or more of the plurality of sets is sampled during a same time period;
receiving, by the one or more computing devices, a plurality of weaning indicators, each of the plurality of weaning indicators corresponding to a respective patient ventilation of the plurality of patient ventilations and the one or more of the plurality of sets sampled during the same time period associated with the respective patient ventilation;
generating, by the one or more computing devices, a trained prediction model based on the received plurality of sets of sampled ventilation parameter values and the received plurality of weaning indicators, the trained prediction model being trained to select, based on an input of ventilation parameter values for a patient ventilation, one or more ventilator parameters having a highest probability, within the group of ventilation parameters, of positively influencing the patient ventilation based on respective threshold values;
receiving, by the one or more computing devices, a plurality of ventilation parameter values sampled during a current patient ventilation;
automatically inputting, by the one or more computing devices, the plurality of ventilation parameter values sampled during the current patient ventilation into the trained prediction model;
receiving, from the trained prediction model, by the one or more computing devices, based on the inputting of the plurality of ventilation parameters, a ventilation parameter selected from the group of ventilation parameters and having the highest probability of positively influencing the current patient ventilation based on a threshold value of the ventilator parameter; and
causing, by the one or more computing devices, an operational mode of a ventilator associated with the current patient ventilation to be adjusted based on the ventilation parameter selected by the trained prediction model.
